(19) United States
(12) Patent Application Publication
(10) Pub. No.: US 2005/0245752 A1
(43) Pub. Date: Nov. 3, 2005

(54) Synthesis of unsymmetric anthracene compounds

(75) Inventors: Scott R. Conley, Rochester, NY (US); Lelia Cosimbescu, Rochester, NY (US); William B. Vreeland, Webster, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/834,826

(22) Filed: Apr. 29, 2004

(51) Int. Cl. C07C 50/16; C07C 17/00

(52) U.S. Cl. 552/271; 570/194

(57) ABSTRACT

A process for forming an unsymmetric anthracene compound comprises a first step of forming a 9-perfluoroalkylsulfonate derivative of anthrone by reacting the anthrone with a perfluoroalkyl sulfonating agent, followed by a second step of contacting the reaction product with an aryl or heteroaryl boronic acid, ester or anhydride, and a palladium catalyst for a period of time sufficient to form an unsymmetric anthracene compound having at least one 9-position aromatic substituent.

### FIELD OF THE INVENTION

This invention relates to the field of organic syntheses and to a process for forming an unsymmetric anthracene compound.

### BACKGROUND OF THE INVENTION

Anthracene compounds have become useful materials and consequently there is a need for synthetic methods that allow their preparation in an economical manner and in high purity. In particular, 9,10-diarylsubstituted anthracene molecules have found use in electroluminescent (EL) devices such as organic light-emitting diodes (OLEDs). For example, U.S. Pat. No. 5,935,721, U.S. Pat. No. 6,465,115, and U.S. 2004/0016907 describe the use of such anthracene derivatives as host materials and as light-emitting materials. It is desirable to use very pure materials in EL devices to ensure long operating lifetimes.

Many of the 9,10-diarylsubstituted anthracene molecules described for use in EL applications have had a symmetrical structure. In this case, a symmetrical structure means that the 9- and 10-substituents are the same. Symmetrical 9,10-diarylanthracene derivatives can be synthesized by various methods, such as by a Suzuki-type metal catalyzed coupling reaction between a halogenated anthracene and an aryl group. A review of useful types of coupling reactions, including the Suzuki coupling reaction, is given by J. Hassan, M. Sevignon, C. Gozzi, E. Schulz, M. Lemaire, Marc, Chem. Rev, 102, 1359 (2002) and references cited therein. For example, reaction of 9,10-dibromoanthracene with at least two equivalents of an arylboronic acid, in the presence of palladium metal, phosphine ligand, and a base, can afford a 9,10-diarylanthracene derivative. This type of synthetic method is described in U.S. Pat. No. 5,935,721. In this case the substituents in the 9,10-positions are identical.

Recently, unsymmetric anthracenes (i.e., those in which the 9- and 10-substituents are not the same) have become of interest, for example see WO 2004/018587, U.S. Ser. No. 10/692,562 and U.S. Ser. No. 10/693,121. Unsymmetric anthracene materials may be made in similar fashion as symmetrical anthracenes but by using a multiple step process. For example, by using the following process:
a) monobromination of anthracene affords 9-bromoanthracene and this intermediate is isolated,
b) reaction of 9-bromoanthracene with one equivalent of an arylboronic acid, in the presence of palladium metal, phosphine ligand, and a base affords a 9-arylanthracene and this intermediate is isolated,
c) bromination of 9-arylanthracene affords 10-bromo-9-arylanthracene and this intermediate is isolated,
d) react of 10-bromo-9-arylanthracene with one equivalent of a different boronic acid, for example aryl boronic acid, in the presence of palladium metal, phosphine ligand, and a base affords an unsymmetric 9-aryl-10-aryl′-anthracene.

Using the process described above, a 9,10-diarylanthracene derivative may be synthesized in which the aryl groups in the 9- and 10-positions are different. However, this synthetic method is not satisfactory because it leads to impure materials. For example, it is very difficult to monobrominate anthracene (step “a” above) and to avoid the formation of 9,10-dibromoanthracene. Once even small amounts of 9,10-dibromoanthracene have formed it is difficult to remove this impurity. If not removed, the 9,10-dibromoanthracene reacts during further steps in the process with arylboronic acid, in the presence of palladium metal, phosphine ligand, and a base (step ‘b’ and step ‘d’) to afford 9,10-diarylanthracene derivatives, which contaminate the reaction mixture further. Consequently if is difficult to prepare unsymmetric anthracenes that have high purity using such a process.

Other methods have been reported to prepare 9-substitued anthracene materials. For example, K. Akiba and coworkers, J. Am. Chem. Soc., 10645 (1999), report reaction of a 9-triflate derivative of anthracene with carbon monoxide, methanol, and a phosphine catalyst to form a 9-carbomethoxy substituted anthracene. However, these methods do not provide a good process to prepare unsymmetric 9,10-diarylanthracenes have high purity.

### SUMMARY OF THE INVENTION

The invention provides a process for forming an unsymmetric anthracene compound comprising a first step of forming a 9-perfluoroalkylsulfonate derivative of anthrone by reacting the anthrone with a perfluoroalkyl sulfonating agent, followed by a second step of contacting the reaction product with an aryl or heteroaryl boronic acid, ester or anhydride, and a palladium catalyst for a period of time sufficient to form an unsymmetric anthracene compound having at least one 9-position aromatic substituent.

The process enables one to prepare unsymmetric 9,10-diarylanthracenes having high purity.

### DETAILED DESCRIPTION OF THE INVENTION

The invention process is summarized above. The process is useful to provide unsymmetric anthracenes, which are anthracenes that do not have the same groups located in both the 9- and 10-positions. It is possible to prepare these materials in high purity.

The reaction process uses an anthrone compound, which may be unsubstituted or substituted. The anthrone compound may be represented by Formula (1), wherein each V represents an independently selected substituent and m is 0-4. Examples of suitable substituents include aryl groups, such as phenyl groups and tolyl groups, and alkyl groups, such methyl groups and t-butyl groups. In one embodiment, adjacent substituents may combine to form fused rings.

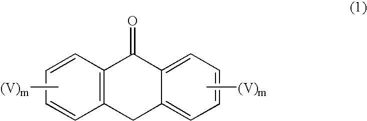

The first step of the process comprises forming a 9-perfluoroalkylsulfonate derivative of anthracene by reacting the anthrone with a suitable perfluoroalkyl sulfonating agent such as a triflating agent. In one embodiment the agent is trifluoromethylsulfonate. The agent can be any reactive material capable of forming a 9-perfluoroalkylsulfonate, such as a sulfonic anhydride or a sulfonyl chloride, for example trifluormethanesulfonic anhydride or trifluorormethanesulfonylchloride. Typically the agent is used at a level of 1 to 3 equivalents relative to the anthrone.

The 9-perfluoroalkylsulfonate derivative of anthracene can be represented by Formula (2), wherein V and m were defined previously and R represents an alkyl group in which the hydrogens were replaced with fluoro groups, that is, a perfluoroalkyl group, of from 1 to 12 carbons.

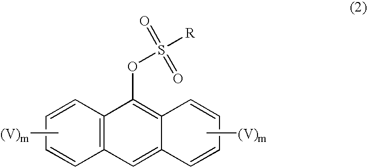

Desirably an acid scavenging base is present during the first step of the process, such as an organic base, for example 1,5-diazabicyclo[4.3.0]undec-7-ene, triethylamine or N,N-diethylisopropylamine or an inorganic base, such as sodium carbonate. Typically the base is used at a level of 1 to 3 equivalents relative to the anthrone.

In one embodiment the first step of the processes is carried out in a solvent. A suitable solvent is one that dissolves the reactants, at least partially, and does not interfere with the reaction. For example, halogenated solvents are useful, such as methylene chloride. Further examples of useful solvents include acetonitrile, aromatic solvents such as benzene and toluene, and ether containing solvents such as tetrahydrofuran.

Desirably the reaction mixture is cooled below room temperature. Typically the reaction is carried out a temperature between −10° C. and 20° C., and often between −5° C. and +5° C.

In one embodiment the anthrone compound is dissolved in the reaction solvent. The reaction mixture is cooled below room temperature, for example by placing it in an ice-water bath. The acid-scavenging base is added and then the perfluoroalkyl sulfonating agent is added. Typically the rates of addition of materials to the reaction mixture are controlled so as to avoid an exotherm, which is a rapid rise of the temperature of the reaction.

Suitable reaction times can be determined by monitoring the reaction. For example, by removing aliquots of the reaction mixture periodically and by using thin-layer-chromatography (TLC) or high-performance-liquid chromatography (HPLC) analysis one can determine the amount of reactants present, e.g. unreacted anthrone, and one can determine the amount of product formed. In this manner the progress of the reaction can be monitored. Typically the reaction times are 1 to 24 h, but may be shorter or longer.

Suitably the 9-perfluoroalkylsulfonate-anthracene derivative may be used without further purification or it may desirably be isolated and purified. Purification can be done by well-known methods such as crystallization or column chromatography.

The second step of the process includes contacting the reaction product with an aryl or heteroaryl boronic acid, ester or anhydride and a palladium catalyst for a period of time sufficient to form an unsymmetric anthracene compound having at least one aromatic substituent in the 9-position. For examples of this type of coupling reaction, including the Suzuki coupling reaction, see J. Hassan, M. Sevignon, C. Gozzi, E. Schulz, M. Lemaire, Marc, Chem. Rev, 102, 1359 (2002) and references cited therein and A. F. Litthe, C. Dai, and G. C. Fu, J. Am. Chem. Soc., 122, 4020 (2000). In one embodiment the boronic acid, ester or anhydride can be represented by Formula (3).

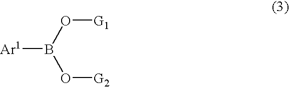

In Formula (3), Ar¹ represents an aromatic group, for example Ar¹ may represent a phenyl group, naphthyl group, pyridyl group or biphenyl group. G₁ and G₂ independently represent hydrogen or a substituent, for example, an alkyl group, alkenyl group or an aryl group. G₁ and G₁ may join together to form a ring group. In one desirable embodiment, G₁ and G₂ represent hydrogen. In this case, the boronic acid may undergo dehydration in solution to form a cyclic anhydride but this does not affect the reaction.

Compounds of Formula (3) are known and may be synthesized or purchased from commercial sources such as Aldrich Chemical Company. Synthetic methods are described in the literature. For example those described in T. Onak, Organoborane Chemistry, Academic Press, New York, 1975.

The palladium catalyst may be derived from a convenient palladium source, for example, palladium halides, including PdCl₂, PdBr₂, palladium carboxylates, including Pd(OAc)₂, Pd(CF₃CO₂)₂ and palladium (II) acetylacetonoate, palladium (II) bis(benzonitrile)dichloride, and tris(dibenzylideneacetone)dipalladium (0). Where Pd(II) ions are derived from these sources, the Pd(II) may be converted to Pd(0) in situ during the course of the process.

The quantity of palladium used in the process is preferably in the range 0.0001 to 10 mole %, more preferably 0.005 to 5 mole %, especially 0.01 to 3 mole %, relative to the quantity of anthracene compound.

In one embodiment, it is desirable to have a phosphine present in the second step of the reaction. Suitable phosphines can be obtained from commercial sources such as Aldrich Chemical Company or synthesized by methods know in the literature. These phosphines are believed to act as ligands to the palladium thereby forming a more effective catalyst for the coupling reaction. In one embodiment, suitable phosphines are substituted by three groups. The groups may be aromatic groups or nonaromatic groups or combinations thereof. In one suitable embodiment the groups include aryl groups such as phenyl groups. Desirably the groups include alkyl groups such as t-butyl groups or cycloalkyl groups. Examples of useful phosphines are triphenylphosphine, tricyclohexylphosphine and tri-t-butylphosphine. Suitable phosphine compounds may comprise more than one phosphine group.

In one embodiment, the quantity of phosphine ligand used in the process may be such that the molar ratio of palladium to phosphorus is from 3 to 0.1, more typically form 1.5 to 0.5 and commonly from 1.1 to 0.9.

In one suitable embodiment, the second step of the process is performed in the presence of a base. The base may be an organic base, such as Na(t-BuO) or K(t-BuO). In one embodiment the base is selected from alkali metal and alkaline earth metal phosphates such as Na₃PO₄ and K₃PO₄. In one embodiment, the quantity of base used in the process may be such that the ratio of equivalents of base to 9-perfluoroalkylsulfonate anthracene derivative is from 3 to 0.1, more typically from 1.5 to 0.5 and commonly from 1.1 to 0.9.

In one embodiment the second step of the process is performed in a reaction solvent or a mixture of solvents. The second step of the process may be carried out in any suitable solvent that dissolves the reactants at least partially and does not interfere with the reaction. Examples of solvents are water and organic solvents, such as hydrocarbons (e.g. toluene or xylene), ethers (e.g. tetrahydrofuran. and diglyme), alcohols, such as aliphatic alcohols, for example ethanol, cyclohexanol. In one embodiment polar aprotic solvents such as N-methylpyrrolidone, dimethylformamide, N,N-dimethylacetamide or dimethylsulphoxide are desirable and mixtures thereof as well as mixtures of aprotic solvent with water.

The second step of the process is preferably performed at a temperature in the range 20° C. to 200° C., typically in the range of 40° C. to 140° C., and commonly in the range of 50° C. to 100° C. Normally atmospheric pressure is used although elevated pressure may be used if desired.

The product obtained from the second step of the process can be represented by Formula (4). Wherein Ar¹, V, m, and n have been defined previously.

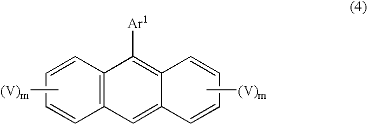

Suitably the anthracene of Formula (4) may used without further purification or, desirably, it may be isolated and purified. Purification can be done by well-known methods such as sublimation, distillation, crystallization or column chromatography.

In one embodiment the unsymmetric anthracene obtained after the second step of the process is reacted further, in a third step, with a halogenating agent to form an unsymmetric anthracene compound substituted with at least one halogen including Cl, Br, or I. In one embodiment the halogen is Br or I. Halogenating agents are well know in the literature, for example, see Norman and Taylor, Electrophilic Substitution in Benzenoid Compounds, American Elseview, New York, 1965 and A. Katritzhy, O. Meth-Cohn, C. Rees, Comprehensive Organic Functional Group Transformations, Vol. 2, New York, Pergamon Press, 1995, pages 619-633. Halogenating agents include such agents as chlorine, bromine, iodine, N-bromosuccimide, N-chlorosuccimide, and 1,3-dibromo-5,5,-dimethylhydantoin. In one desirable embodiment X represents Br and the halogenating agent is 1,3-dibromo-5,5,-dimethylhydantoin. To facilitate the third step of the process it may be desirable to expose the reaction mixture to UV or visible light or to add a catalyst, such as a Lewis acid, for example, ferric chloride or thallium acetate.

The third step of the process may be carried out in a solvent. Suitable solvents dissolve the reactants, at least partially, and do not adversely react with the components of the reaction mixture. Examples of useful solvents include alkanes and halogenated alkanes, such as methylene chloride. Desirably, the amount of halogenating agent is present in an amount sufficient to substantially monohalogenate the anthracene derivative. Typically the halogenating agent amount is in the range of 0.8 to 2.0 equivalents and often in the range of 0.9 to 1.2 equivalents.

The product obtained from the third step of the process can be represented by Formula (5). Wherein Ar¹, V, m, and n have been defined previously, and X represents a halogen, including Cl, Br, or I. Suitably the anthracene of Formula (5) may used without further purification or, desirably, it may be isolated and purified. Purification may be done by well-known methods such as sublimation, distillation, crystallization or column chromatography.

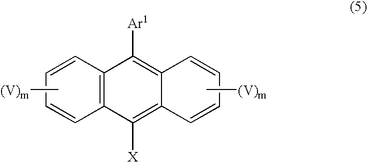

In one embodiment, the unsymmetric anthracene compound of Formula (5) is reacted in a fourth step, with an aryl or heteroaryl boronic acid of Formula (6) and a palladium catalyst for a period of time sufficient to form an unsymmetric anthracene compound with aromatic substituents in the 9- and 10-positions. For examples of this type of coupling reaction, including the Suzuki coupling reaction, see J. Hassan, M. Sevignon, C. Gozzi, E. Schulz, M. Lemaire, Marc, Chem. Rev, 102, 1359 (2002) and references cited therein and M. Montieth, EP 0934236.

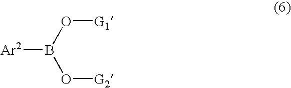

In Formula (6), Ar² represents an aromatic group, for example, Ar² may be a phenyl group, naphthyl group, pyridyl group or biphenyl group. G₁′ and G₂′ represent hydrogen or an independently selected substituent, for example, an alkyl group, alkenyl group or an aryl group. G₁′ and G₂′ may join together to form a ring group. In one desirable embodiment, G₁′ and G₂′ represent hydrogen. In this case, the boronic acid may undergo dehydration in solution to form a cyclic anhydride but this does not affect the reaction. In one embodiment Ar² does not represent the same group as Ar¹ of Formula (5).

As described previously for Formula (3), compounds of Formula (6) are known and may be synthesized or purchased from commercial sources such as Aldrich Chemical Company. Synthetic methods are described in the literature. For example those described in T. Onak, Organoborane Chemistry, Academic Press, New York, 1975.

The palladium catalyst may be derived from a convenient palladium source, suitable palladium sources and their quantities for use in the fourth step have been described previously for the second step of the process.

In one embodiment it is desirable to have a phosphine present in the second step of the reaction. Suitable phosphines for use in the fourth step and their quantities have been described previously for the second step of the process.

In one suitable embodiment, the fourth step of the process is performed in the presence of a base. Suitable bases for use in the fourth step and their quantities have been described previously for the second step of the process.

Suitable solvents and reaction times for the fourth step of the process have been described previously for the second step of the process.

The product obtained from the fourth step of the process may be represented by Formula (7).

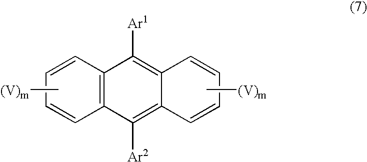

In Formula (7), Ar¹, V, m, and n have been defined previously, and Ar² represents an aryl or heteroaryl group. Desirably, Ar¹ and Ar² do not represent the same group. Suitably Ar¹ and Ar² represent different aryl groups. In one desirable embodiment, Ar¹ represents a naphthyl group and Ar² represents a biphenyl group.

Illustrative examples of compounds of Formula (7) are listed below.

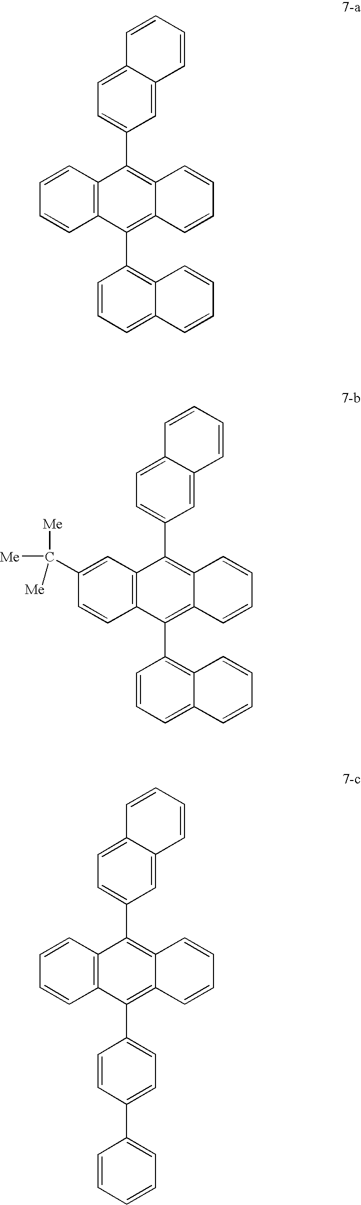

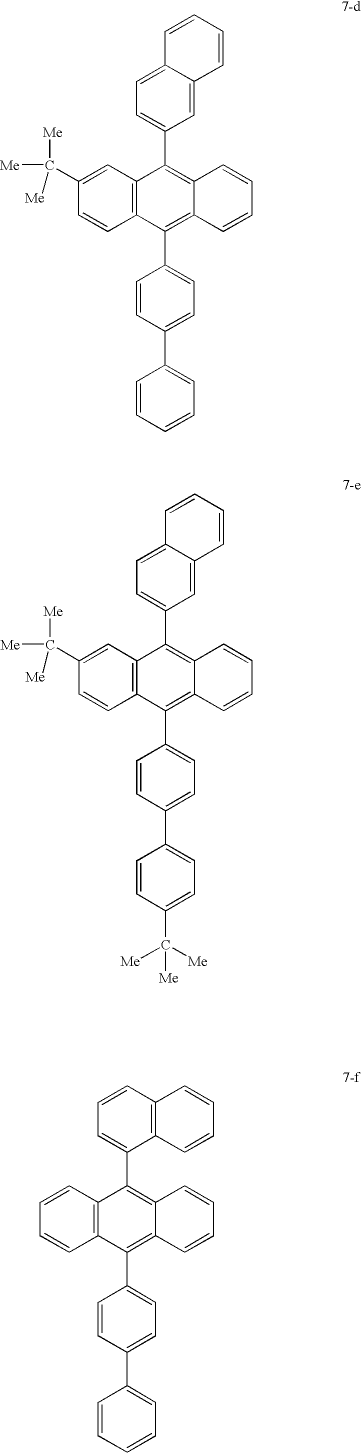

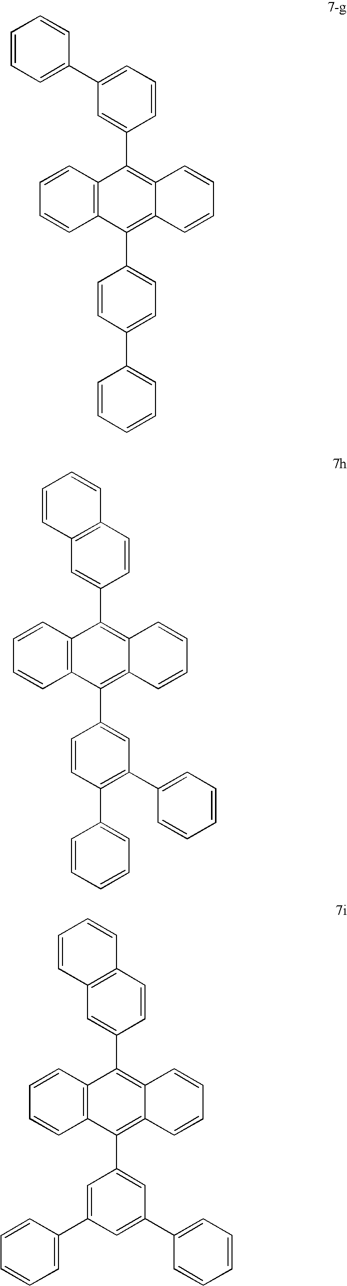

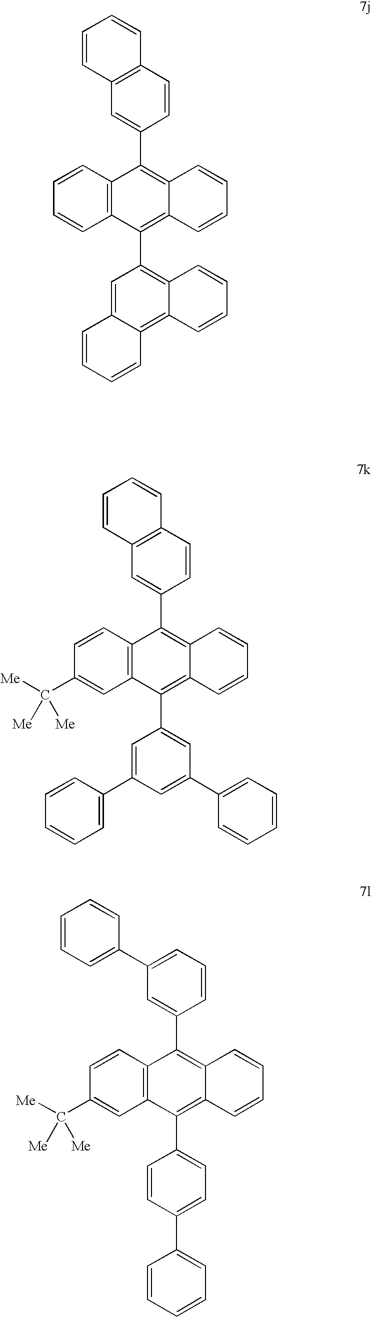

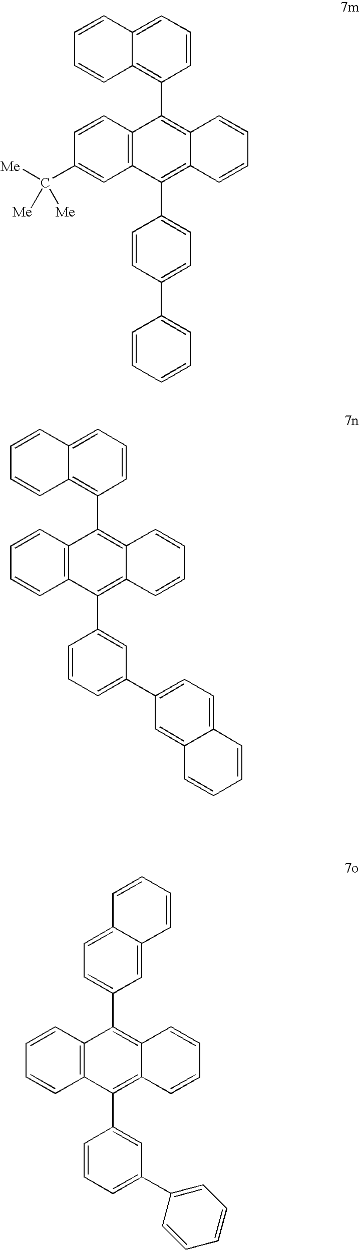

Unless otherwise specifically stated, use of the term “substituted” or “substituent” means any group or atom other than hydrogen. Additionally, when the term “group” is used, it means that when a substituent group contains a substitutable hydrogen, it is also intended to encompass not only the substituent's unsubstituted form, but also its form further substituted with any substituent group or groups as herein mentioned, so long as the substituent does not destroy properties necessary for device utility. Suitably, a substituent group may be halogen or may be bonded to the remainder of the molecule by an atom of carbon, silicon, oxygen, nitrogen, phosphorous, sulfur, selenium, or boron. The substituent may be, for example, halogen, such as chloro, bromo or fluoro; nitro; hydroxyl; cyano; carboxyl; or groups which may be further substituted, such as alkyl, including straight or branched chain or cyclic alkyl, such as methyl, trifluoromethyl, ethyl, t-butyl, 3-(2,4-di-t-pentylphenoxy) propyl, and tetradecyl; alkenyl, such as ethylene, 2-butene; alkoxy, such as methoxy, ethoxy, propoxy, butoxy, 2-methoxyethoxy, sec-butoxy, hexyloxy, 2-ethylhexyloxy, tetradecyloxy, 2-(2,4-di-t-pentylphenoxy)ethoxy, and 2-dodecyloxyethoxy; aryl such as phenyl, 4-t-butylphenyl, 2,4,6-trimethylphenyl, naphthyl; aryloxy, such as phenoxy, 2-methylphenoxy, alpha- or beta-naphthyloxy, and 4-tolyloxy; carbonamido, such as acetamido, benzamido, butyramido, tetradecanamido, alpha-(2,4-di-t-pentylphenoxy)acetamido, alpha-(2,4-di-t-pentylphenoxy)butyramido, alpha-(3-pentadecylphenoxy)-hexanamido, alpha-(4-hydroxy-3-t-butylphenoxy)tetradecanamido, 2-oxo-pyrrolidin-1-yl, 2-oxo-5-tetradecylpyrrolin-1-yl, N-methyltetradecanamido, N-succinimido, N-phthalimido, 2,5-dioxo-1-oxazolidinyl, 3-dodecyl-2,5-dioxo-1-imidazolyl, and N-acetyl-N-dodecylamino, ethoxycarbonylamino, phenoxycarbonylamino, benzyloxycarbonylamino, hexadecyloxycarbonylamino, 2,4-di-t-butylphenoxycarbonylamino, phenylcarbonylamino, 2,5-(di-t-pentylphenyl)carbonylamino, p-dodecylphenylcarbonylamino, p-tolyl carbonyl amino, N-methylureido, N,N-dimethylureido, N-methyl-N-dodecylureido, N-hexadecylureido, N,N-dioctadecylureido, N,N-dioctyl-N′-ethylureido, N-phenylureido, N,N-diphenylureido, N-phenyl-N-p-tolylureido, N-(m-hexadecylphenyl)ureido, N,N-(2,5-di-t-pentylphenyl)-N′-ethylureido, and t-butylcarbonamido; sulfonamido, such as methylsulfonamido, benzenesulfonamido, p-tolylsulfonamido, p-dodecylbenzenesulfonamido, N-methyltetradecylsulfonamido, N,N-dipropylsulfamoylamino, and hexadecylsulfonamido; sulfamoyl, such as N-methylsulfamoyl, N-ethylsulfamoyl, N,N-dipropylsulfamoyl, N-hexadecylsulfamoyl, N,N-dimethylsulfamoyl, N-[3-(dodecyloxy)propyl]sulfamoyl, N-[4-(2,4-di-t-pentylphenoxy)butyl]sulfamoyl, N-methyl-N-tetradecylsulfamoyl, and N-dodecylsulfamoyl; carbamoyl, such as N-methylcarbamoyl, N,N-dibutylcarbamoyl, N-octadecylcarbamoyl, N-[4-(2,4-di-t-pentylphenoxy)butyl]carbamoyl, N-methyl-N-tetradecylcarbamoyl, and N,N-dioctylcarbamoyl; acyl, such as acetyl, (2,4-di-t-amylphenoxy)acetyl, phenoxycarbonyl, p-dodecyloxyphenoxycarbonyl methoxycarbonyl, butoxycarbonyl, tetradecyloxycarbonyl, ethoxycarbonyl, benzyloxycarbonyl, 3-pentadecyloxycarbonyl, and dodecyloxycarbonyl; sulfonyl, such as methoxysulfonyl, octyloxysulfonyl, tetradecyloxysulfonyl, 2-ethylhexyloxysulfonyl, phenoxysulfonyl, 2,4-di-t-pentylphenoxysulfonyl, methylsulfonyl, octylsulfonyl, 2-ethylhexylsulfonyl, dodecylsulfonyl, hexadecylsulfonyl, phenylsulfonyl, 4-nonylphenylsulfonyl, and p-tolylsulfonyl; sulfonyloxy, such as dodecylsulfonyloxy, and hexadecylsulfonyloxy; sulfinyl, such as methylsulfinyl, octylsulfinyl, 2-ethylhexylsulfinyl, dodecylsulfinyl, hexadecylsulfinyl, phenylsulfinyl, 4-nonylphenylsulfinyl, and p-tolylsulfinyl; thio, such as ethylthio, octylthio, benzylthio, tetradecylthio, 2-(2,4-di-t-pentylphenoxy)ethylthio, phenylthio, 2-butoxy-5-t-octylphenylthio, and p-tolylthio; acyloxy, such as acetyloxy, benzoyloxy, octadecanoyloxy, p-dodecylamidobenzoyloxy, N-phenylcarbamoyloxy, N-ethylcarbamoyloxy, and cyclohexylcarbonyloxy; amine, such as phenylanilino, 2-chloroanilino, diethylamine, dodecylamine; imino, such as 1 (N-phenylimido)ethyl, N-succinimido or 3-benzylhydantoinyl; phosphate, such as dimethylphosphate and ethylbutylphosphate; phosphite, such as diethyl and dihexylphosphite; a heterocyclic group, a heterocyclic oxy group or a heterocyclic thio group, each of which may be substituted and which contain a 3 to 7 membered heterocyclic ring composed of carbon atoms and at least one hetero atom selected from the group consisting of oxygen, nitrogen, sulfur, phosphorous, or boron. such as 2-furyl, 2-thienyl, 2-benzimidazolyloxy or 2-benzothiazolyl; quaternary ammonium, such as triethylammonium; quaternary phosphonium, such as triphenylphosphonium; and silyloxy, such as trimethylsilyloxy.

If desired, the substituents may themselves be further substituted one or more times with the described substituent groups. The particular substituents used may be selected by those skilled in the art to attain the desired desirable properties for a specific application and can include, for example, electron-withdrawing groups, electron-donating groups, and steric groups. When a molecule may have two or more substituents, the substituents may be joined together to form a ring such as a fused ring unless otherwise provided. Generally, the above groups and substituents thereof may include those having up to 48 carbon atoms, typically 1 to 36 carbon atoms and usually less than 24 carbon atoms, but greater numbers are possible depending on the particular substituents selected.

The invention and its advantages can be better appreciated by the following examples.

### EXAMPLE 1

### Preparation of 9-biphenyl-10-(2-naphthyl)anthracene

Step 1 of the process can be illustrated by the preparation of 9-trifluoromethanesulfonyloxyanthracene in the following manner. As 1,5-diazabicyclo[4.3.0]undec-7-ene (DBU, 30.9 mmol, 3.1 mL) was added slowly to a solution of anthrone (10.3 mmol, 2 g) in methylene chloride (50 mL) at 0° C., the mixture turned orange. Trifluoromethanesulfonic anhydride (11.3 mmol, 1.9 mL) was added to the mixture slowly. Water was added after stirring at room temperature for 4 hours. The mixture was extracted with methylene chloride twice, then the organic layers were combined and dried with magnesium sulfate, filtered, and concentrated under vacuum. Purification by column chromatography (10% EtOAc/5% CH₂Cl₂/85% heptane) gave 1.8 g (55% yield) of pure 9-trifluoromethanesulfonyloxyanthracene.

Step 2 of the process can be illustrated by the preparation 9-(2-naphthyl)anthracene in the following manner. 9-Trifluoromethanesulfonyloxyanthracene (1.935 mmol, 0.63 g), 2-naphthylboronic acid (2.36 mmol, 0.41 g), and K₃PO₄ (8.58 mmol, 1.8 g) were stirred in degassed NMP (N-methylpyrrolidinone, 4 mL) and water (2.8 mL) at 50° C. A mixture of Pd(OAc)₂ (0.06 mmol, 0.04 g), tricyclohexylphosphine (0.06 mmol, 0.018 g), and 9-trifluoromethanesulfonyloxyanthracene (0.215 mmol, 0.07 g) was stirred for 10 min. then added to the boronic acid mixture. After 4 h the mixture was extracted with methylene chloride and filtered through celite. Purification by column chromatography (7% EtOAc/93% heptane) gave 0.64 g (98% yield) of pure 9-(2-naphthyl)anthracene.

Step 3 of the process can be illustrated by the preparation of 9-bromo-10-(2-naphthyl)anthracene in the following manner. 9-(2-Naphthyl)anthracene (14 g, 48 mmol, 1 eq) and N-bromosuccinimide (8.9 g, 50 mmol, 1.05 eq) were combined with 140 ml CH₂Cl₂ in a 500 ml round bottom flask. The mixture was stirred at room temperature under nitrogen in the presence of light from a 100 W lamp and the mixture quickly became homogeneous. Reaction was complete after 3 h, as indicated by TLC (ligroin:CH₂Cl₂/9:1). Approximately half the solvent volume was evaporated under reduced pressure until solid started to precipitate from the reaction mixture. Then, enough acetonitrile was added with heating to dissolve the solid. Additional CH₂Cl₂ was then evaporated under reduced pressure just to the point that solid again began to precipitate. The solution was cooled and left to crystallize. The resulting solid was isolated by filtration, washed with a small amount of acetonitrile, and dried to yield 17 g (92%) of 9-bromo-10-(2-naphthyl)anthracene.

Step 4 of the process can be illustrated by the preparation of 9-biphenyl-10-(2-naphthyl)anthracene. 9-Bromo-10-(2-naphthyl)anthracene (8.9 g, 23 mmol, 1 eq), 4-biphenylboronic acid (4.8 g, 24 mmol, 1.05 eq) together with (PPh₃)₂PdCl₂ (0.16 g, 0.7 eq %) were combined in 200 mL toluene in a 500 mL round bottom flask, and the resulting suspension was sonicated for 30 min. Then the mixture was quickly heated to reflux, and the solution became homogeneous after 15 min of reflux. After 2 h, solid started to precipitate out of the reaction mixture, while the mixture remained light yellow (indicative of the catalyst being active). The mixture was refluxed overnight. The mixture was filtered hot, through a glass fiber filter paper to remove precipitated solid. The solid was re-dissolved in about 1 L of hot toluene and the solution was filtered through a glass fiber filter paper to remove palladium impurities. The filtrate was concentrated to yield 9.2 g of off-white solid. From the original filtrate that contained the aqueous layer, the toluene layer was isolated, washed with H₂O, dried with saturated brine solution, dried over MgSO₄, concentrated and crystallized to yield 1.3 g of solid. Both solid batches were combined to yield 10.4 g of clean product (98% yield). The material was sublimed at 260° C. to yield a very pure fraction (8.26 g, 99.8% assay) and a pure fraction (1.45 g, 99.3% assay).

### COMPARISON EXAMPLE 1

As discussed previously unsymmetric anthracene materials may be made alternatively by using a multiple step process such as:
a) monobromination of anthracene affords 9-bromoanthracene,
b) reaction of 9-bromoanthracene with one equivalent of an arylboronic acid, in the presence of palladium metal, phosphine ligand, and a base affords a 9-arylanthracene,
c) after isolation, bromination of this 9-arylanthracene affords 10-bromo-9-arylanthracene,
d) after isolation, reaction of 10-bromo-9-arylanthracene with one equivalent of a different boronic acid, for example aryl boronic acid, in the presence of palladium metal, phosphine ligand, and a base may afford a 9-aryl-10-aryl′-anthracene.

In this alternative process, Step (a) includes the preparation of 9-bromoanthracene by the bromination of anthracene. In an attempt to keep the 9,10-dibromoanthracene by product to a minimum, the anthracene was under-brominated by the following procedure wherein a slight excess of anthracene was used relative the brominating agent.

Anthracene (50 g, 0.28 mol, 1 eq) and 1,3-dibromo-5,5-dimethylhydantoin (38.15 g, 0.133 mol, 0.475 eq) were combined in a 500 mL of ethyl acetate and brought to reflux. After about 5 min of reflux, the mixture became homogeneous, and yellow, and stayed light in color throughout the 2.5 h that it was refluxed. The reaction mixture allowed to cool and then washed with water to remove the 5,5-dimethylhydantoin. When water was added, the mixture turned greenish-blue and the color persisted through additional aqueous washes. The organic layer was dried over Na₂SO₄ and the greenish color gradually disappeared, and the mixture became orange-brown. The liquor was concentrated to dryness, to yield a brown solid residue. The solid was dissolved in hot THF (about 50 mL) and then slowly it was precipitated again with addition of acetonitrile (about 200 mL). The yellowish solid was isolated by filtration (36.4 g) and the process was repeated twice to yield two more crops of solid (total 23.8 g). The 3 crops were combined and analyzed by high performance liquid-chromatography (HPLC). The analysis indicated that only 89% of the reaction product was the desired 9-bromoanthracene, 9.3% was unreacted anthracene and 1% was the undesirable 9,10-dibromoanthracene. The 9,10-dibromoanthracene could not be removed by recrystallization.

In the alternative process Step (b) includes preparation of 9-arylanthracene. This is illustrated for the preparation of 9-naphthylanthracene by the following procedure. 9-Bromoanthracene (5 g, 19.4 mmol, 1 eq, prepared by the procedure described above) was combined with 2-naphthylboronic acid (3 g, 17.5 mmol, 0.9 eq) in 30 mL toluene: The resulting suspension was degassed for 10 min under N₂, then catalyst was added (0.5 mole %, 68 mg) followed by the addition of 10 mL of 2M Na₂CO₃ solution. The mixture was quickly heated to reflux, and stirred for 3 h (analysis by thin-layer chromatography (TLC) indicated the reaction was complete). The mixture was filtered hot through glass fiber filter paper, to remove Pd. The organic filtrate was separated, washed with water (twice with 25 mL) then dried over Na₂SO₄. The toluene extract was concentrated and left to crystallize slowly overnight. The solid formed was collected (3.37 g) and its purity solid was determined by HPLC analysis. Analysis indicated that 94% of the solid was the desired product, 9-naphthylanthracene. Three major impurities were present, including 1.2% of unreacted starting material, 9-bromoanthracene. There was 2.3% anthracene present. The presence of anthracene creates a problem because the next step in this alternative process, step ‘c’, involves a bromination and anthracene will brominate in both the 9- and 10-positions leading to further impurities. There was 1.3% of 9,10-di-(2-naphthyl)anthracene. This impurity is very undesirable because it is extremely difficult to remove. An attempt to remove 9,10-dinaphthylanthracene by recrystallization from heptane was unsuccessful and it continued to be present at a level of 1%.

It can be seen from Example 1 that the process according to the invention affords a method of making unsymmetric anthracenes in high purity. Other synthetic methods, such as the process described in Comparison Example 1, do not provide materials in sufficiently high purity.

The unsymmetric anthracene materials synthesized according to this invention may be incorporated in an EL device. In one embodiment the unsymmetric anthracene materials are included in an emissive layer.

The entire contents of the patents and other publications referred to in this specification are incorporated herein by reference. The invention has been described in detail with particular reference to certain preferred embodiments thereof, but it will be understood that variations and modifications can be effected within the spirit and scope of the invention.

1. A process for forming an unsymmetric anthracenyl compound comprising a first step of forming an unsymmetric 9-anthracenyl perfluoroalkylsulfonate compound by reacting an anthrone with a perfluoroalkyl sulfonating agent, followed by a second step of contacting the reaction product with an aryl or heteroaryl boronic acid, ester or anhydride, and a palladium catalyst for a period of time sufficient to form an unsymmetric anthracenyl ompound having at least one 9-position aromatic substituent.

2. The process of claim 1 wherein the anthrone is represented by Formula (1), 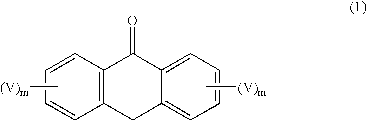
wherein:
V represents an independently selected substituent, provided adjacent substituents may combine to form fused rings; and
each m is independently 0-4.

3. The process of claim 1 wherein a 9-anthracenyl trifluoromethylsulfonate compound is formed as an intermediate.

4. The process of claim 1 wherein the 9-anthracenyl perfluoromethanesulfonate compound is isolated in the first step before reacting it with an aryl or heteroaryl boronic acid and a palladium catalyst in the second step.

5. The process of claim 1 wherein the perfluoroalkyl sulfonating agent is a perfluoroalkylsulfonate anhydride.

6. The process of claim 1 wherein a base is present in the first step.

7. The process of claim 1 wherein the 9-anthracenyl perfluoroalkylsulfonate compound is represented by Formula (2), 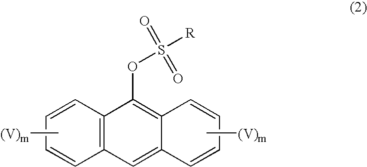
wherein
V represents an independently selected substituent, provided adjacent substituents may combine to form fused rings;
each m is independently 0-4; and
R represents a perfluoroalkyl group, of from 1 to 12 carbons.

8. The process of claim 1 wherein a base is present in the second step.

9. The process of claim 1 wherein a phosphine ligand is present in the second step.

10. The process of claim 9 wherein the phosphine ligand is substituted with alkyl groups.

11. The process of claim 9 wherein the phosphine ligand is substituted with t-butyl or cyclohexyl groups.

12. The process of claim 1 wherein the aryl or heteroaryl boron compound comprises a compound of Formula (3): 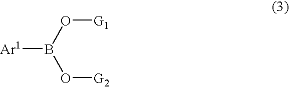
wherein:
Ar¹ represents an aromatic group; and
G₁ and G₂ represent independently selected substituents provided G₁ and G₂ may combine to form a ring.

13. The process of claim 12 wherein Ar₁ represents a naphthyl group or a biphenyl group.

14. The process of claim 1 wherein the unsymmetric anthracene compound formed comprises a compound of Formula (4): 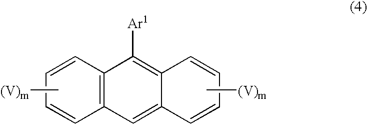
wherein:
V represents an independently selected substituent, provided adjacent substituents may combine to form fused rings;
each m is independently 0-4; and
Ar¹ represents an aromatic group.

15. The process of claim 1 wherein the palladium catalysis comprises palladium in the oxidation state of (II).

16. The process of claim 13 wherein the palladium catalysis comprises palladium acetate.

17. The process of claim 1 wherein the unsymmetric anthracenyl compound is reacted further, in a third step, with a halogenating agent to form an unsymmetric anthracenyl compound substituted with at least one halogen.

18. The process of claim 17 wherein the anthracenyl compound substituted with at least one halogen comprises a compound of Formula (5): 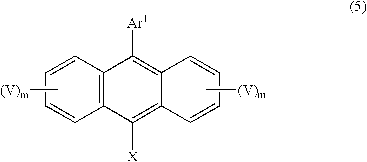
wherein:
V represents an independently selected substituent, provided adjacent substituents may combine to form fused rings;
each m is independently 0-4;
Ar¹ represents an aromatic group; and
X represents Cl, Br or I.

19. The process of claim 17 wherein the unsymmetric anthracenyl compound substituted with at least one halogen is reacted, in a fourth step, with an aryl or hetroaryl boronic acid and a palladium catalyst for a period of time sufficient to form an unsymmetric 9,10-disubstituted anthracenyl compound wherein the substituents in the 9- and 10-positions are different.

20. The process of claim 19 wherein the unsymmetric 9,10-disubstituted anthracenyl compound comprises a compound of Formula (7): 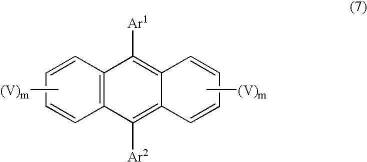
wherein:
V represents an independently selected substituent, provided adjacent substituents may combine to form fused rings;
m is 0-4;
Ar¹ and Ar² represent independently selected aromatic groups, provided Ar¹ and Ar² do not represent the same group.

21. The process of claim 20 wherein Ar¹ and Ar² represent independently selected aryl groups.

22. The process of claim 20 wherein Ar¹ and Ar² represent independently selected naphthyl or biphenyl groups.

23. The process of claim 19 wherein a base is present during the fourth step.

24. The process of claim 19 wherein the palladium catalysis of the fourth step comprises palladium in the oxidation state of (II).
